(19) United States
(12) Patent Application Publication
(10) Pub. No.: US 2018/0208550 A1
(43) Pub. Date: Jul. 26, 2018

(54) METHOD FOR PRODUCING A CYCLIC ISOCYANATE

(71) Applicant: BASF SE, Luwigshafen (DE)

(72) Inventors: Klaus BREUER, Speyer (DE); Robert BAUMANN, Mannheim (DE); Alexander PANCHENKO, Ludwigshafen (DE); Michael LORENZ, Ludwigshafen (DE); Frank HETTCHE, Weinheim (DE); Norbert GUTFRUCHT, Lambrecht (DE); Thomas FENLON, Mannheim (DE)

(73) Assignee: BASF SE, Luwigshafen (DE)

(21) Appl. No.: 15/746,151

(22) PCT Filed: Jul. 20, 2016
(86) PCT No.: PCT/EP2016/067254

§ 371 Date: Jan. 19, 2018

(30) Foreign Application Priority Data
Jul. 21, 2015 (EP) 15177628.3

(51) Int. Cl. C07C 263/10

(52) U.S. Cl. CPC C07C 263/10; C07B 2200/09; C07C 2601/14

(57) ABSTRACT

The present invention relates to a process for preparing a cyclic isocyanate (B) from a composition (Z) which comprises at least one component (A) which has at least one cycloalkane ring having at least 4 ring carbons, where the cycloalkane ring has two NH₂ groups in β or γ positions relative to one another as substituents and in more than 50 mol % of the total amount of component (A) the two NH₂ groups in β or in γ positions relative to one another assume a trans configuration relative to one another. This composition (Z) is, in the process of the invention, reacted with phosgene to give a composition (ZP) which comprises at least one cyclic isocyanate (B) having isocyanate groups.

In addition, the invention relates to the use of the isocyanate mixture (M) as monomer in processes for preparing polymers, in particular for preparing polyurethanes and polyureas.

The present invention relates to a process for preparing a cyclic isocyanate (B) from a composition (Z) which comprises at least one component (A) which has at least one cycloalkane ring having at least 4 ring carbons, where the cycloalkane ring has two NH₂ groups in β or γ positions relative to one another as substituents and in more than 50 mol % of the total amount of component (A) the two NH₂ groups in β or in γ positions relative to one another assume a trans configuration relative to one another. This composition (Z) is, in the process of the invention, reacted with phosgene to give a composition (ZP) which comprises at least one cyclic isocyanate (B) having isocyanate groups.

In addition, the invention relates to the use of the isocyanate mixture (M) as monomer in processes for preparing polymers, in particular for preparing polyurethanes and polyureas.

The preparation of isocyanates from amines by direct phosgenation is a process which is well established in the prior art.

Thus, for example, US 2013/0060062 A1 describes a process for preparing isocyanates in the liquid phase and in the gas phase.

However, the production of cycloaliphatic 1,2- and 1,3-diisocyanates in economically feasible yields is still a technical challenge. The reason for the reduced yields is secondary reactions which can occur, especially in the case of cycloaliphatic 1,2- and 1,3-diamines as starting material. Thus, U.S. Pat. No. 3,351,650 discloses the direct phosgenation of a mixture of 2,4- and 2,6-diamino-1-methylcyclohexane, but a yield of only 21.2% could be achieved here.

In DE-A 2005309, the reaction of 1,3-cyclohexanediamines with a mixture of phosgene and hydrogen chloride, which results in increased yields of the corresponding 1,3-di-isocyanates, is proposed as a possible solution to this problem.

EP 0676392 A1 discloses a gas-phase phosgenation for preparing diisocyanates starting out from aliphatic or cycloaliphatic diamines having amino groups in the 1,2 or 1,3 positions relative to one another. In the continuous process, the gaseous diamines diluted with an inert gas or with the vapor of an inert solvent are reacted with phosgene at temperatures of from 200 to 600° C. Under these conditions, significantly increased yields of the corresponding diisocyanates were obtained.

EP 0928785 A1 describes a process for the phosgenation of (cyclo)aliphatic diamines in the gas phase, comprising, inter alia, diamines whose amino groups can be in 1,3 positions relative to one another. Increased yields, due to the decrease in by-products, are obtained here by rapid and efficient mixing of starting material and phosgene by means of microstructural mixers.

EP 0289840 A1 discloses a general process for preparing (cyclo)aliphatic diisocyanates by phosgenation of diamines in the gas phase. Although the phosgenation of (cyclo)aliphatic diamines having amino groups in the 1,2 or 1,3 positions relative to one another is encompassed, no specific measures for reducing by-product formation and/or increasing the yield specifically in the case of these substrates are described.

It is therefore an object of the present invention to provide a process for preparing a cyclic isocyanate.

This object is achieved by a process for preparing a cyclic isocyanate (B), comprising the steps a) and b):

a) provision of a composition (Z) which comprises at least one component (A) which has at least one cycloalkane ring having at least 4 ring carbons, where the cycloalkane ring has two NH₂ groups in β or γ positions relative to one another as substituents and in more than 50 mol % of the total amount of component (A) the two NH₂ groups in β or γ positions relative to one another assume a trans configuration relative to one another and

b) reaction of the composition (Z) with phosgene to give a composition (ZP) which comprises at least one cyclic isocyanate (B) having two isocyanate groups.

It has surprisingly been found that the formation of by-products in the direct reaction of cycloaliphatic diamines in which the NH₂ groups are in β or γ positions, preferably γ position, relative to one another with phosgene to form isocyanates can be significantly reduced when more than 50 mol % of these amines have the NH₂ groups in a trans configuration relative to one another.

In contrast to the known processes described in the prior art discussed above, the direct phosgenation of the amines to form isocyanates with a reduction in by-product formation can now not only necessarily be carried out in the gas phase but also at significantly lower temperatures.

The invention will be described in detail below.

The invention provides a process for preparing a cyclic isocyanate (B). For the purposes of the present invention, the cyclic isocyanate (B) is a compound which has at least one cycloalkane ring and at least two isocyanate groups. Such compounds are known to those skilled in the art

The process comprises the steps a) and b).

Step a) comprises provision of a composition (Z) which comprises at least one component (A) which has at least one cycloalkane ring having at least 4 ring carbons, where the cycloalkane ring has two NH₂ groups in β or γ positions relative to one another as substituents and in more than 50 mol % of the total amount of component (A) the two NH₂ groups in β or γ positions relative to one another assume a trans configuration.

This composition (Z) comprises at least one component (A).

The component (A) preferably comprises a plurality of structurally different subcomponents which individually correspond to the definition of the component (A).

The component (A) has at least one cycloalkane ring having at least 4 ring carbons. The cycloalkane ring has two NH₂ groups in β or γ positions, preferably γ positions, relative to one another as substituents.

The proportion of the component (A) preferably makes up at least 90% by weight, more preferably at least 95% by weight, particularly preferably at least 98% by weight, of the total amount of the composition (Z).

For the purposes of the present invention, in β or γ positions means a particular relative distance in terms of the number of ring carbons between the NH₂ groups bound as substituents to the cycloalkane ring. In the case of β positions, the distance between the NH₂ groups relative to one another is two carbon atoms, as shown in the formulae (IV), (V) and (VI) below for possible embodiments of the invention.

In the case of γ positions, the distance between the NH₂ groups is three ring carbons, as shown in the formulae (I), (II) and (Ill) below for possible embodiments of the invention.

In more than 50 mol %, preferably more than 60 mol %, even more preferably more than 65 mol %, particularly preferably in more than 70 mol %. of the at least one component (A), based on the total amount of component (A), the two NH₂ groups in β or γ positions relative to one another assume a trans configuration relative to one another.

In a further embodiment, in from 60 to 98 mol %, preferably from 65 to 95 mol %, even more preferably from 70 to 90 mol %, of the at least one component (A), based on the total amount of component (A), the two NH₂ groups in β or γ positions relative to one another assume a trans configuration relative to one another.

Component (A) is preferably selected from among one of the compounds having the formulae (I), (II), (Ill), (IV), (V) or (VI)

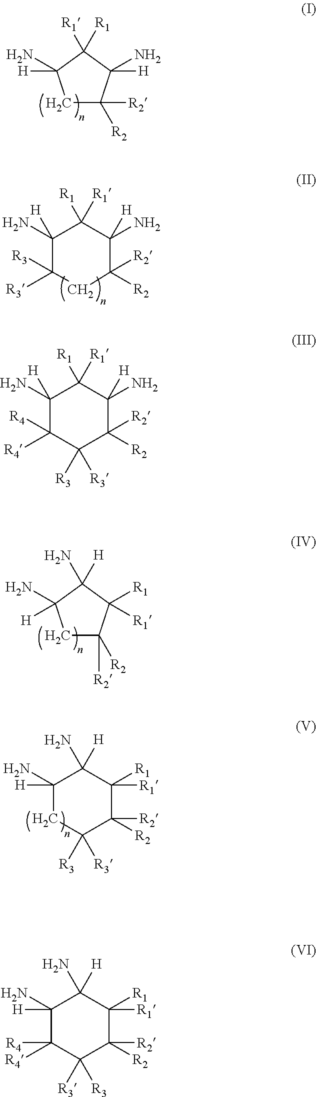

where
n is from 0 to 10,
R₁, R₁′, R₂, R₂′, R₃, R₃′, R₄, R₄′ are each H, C₁-C₁₂-alkyl, C₂-C₁₀-alkenyl, aryl, arylalkyl or —OR₅;
R₅is C₁-C₁₂-alkyl,
where the substituents R₁, R₁′, R₂, R₂′, R₃, R₃′, R₄ , R₄′ are identical or are selected independently of one another.

Even more preferably, component (A) is selected from among one of the compounds having the formulae (I), (II), (III), (IV), (V) or (VI), where

n is from 0 to 5,

R₁, R₁′, R₂, R₂′, R₃, R₃′, R₄, R₄′ are each H, CᵣC₅-alkyl, C₂-C₆-alkenyl, aryl, arylalkyl, or —OR₅

R₅ is C₁C₂-alkyl.

The substituents R₁, R₁′, R₂, R₂′, R₃, R₃′, R₄, R₄′ are identical or are selected independently of one another.

The component (A) is even more preferably selected from among one of the compounds having the formulae (I), (II) or (III).

For the purposes of the present invention, definitions such as C₁-C₁₂-alkyl, as defined, for example, for the radical R₁ in formula (I), mean that this substituent can be an alkyl radical having from 1 to 12 carbon atoms. It can be linear, branched or cyclic, or else at the same time have, in parts, all three forms. Examples of alkyl radicals are methyl, ethyl, propyl, butyl, pentyl, hexyl, heptyl, cyclohexyl, octyl, nonyl or decyl.

For the purposes of the present invention, definitions such as C₂-C₁₀-alkenyl, as defined, for example, for the radical R₁ in formula (I), mean that this substituent (radical) is an alkenyl radical having from 2 to 10 carbon atoms. This carbon radical is preferably monounsaturated, but can optionally also be doubly or multiply unsaturated. As regards linearity, branching and cyclic parts, what has been said above in respect of the C₁-C₁₂-alkyl radicals applies analogously. C₂-C₁₀-Alkenyl is, for the purposes of the present invention, preferably vinyl, 1-allyl, 3-allyl, 2-allyl, cis- or trans-2-butenyl, ω-butenyl.

For the purposes of the present invention, the term “aryl” as, for example, defined above for the radical R₁ in formula (I) means that the substituent (radical) is an aromatic. The aromatic can be a monocyclic, bicyclic or optionally polycyclic aromatic. In the case of polycyclic aromatics, individual rings can optionally be fully or partially saturated. Preferred examples for aryl are phenyl, naphthyl or anthracyl, in particular phenyl.

For the purposes of the present invention, the term “arylalkyl” as, for example, defined for the radical R₁ in formula (I) means that this substituent (radical) is an alkyl radical which is terminally substituted by an aromatic. The alkyl radical in the arylalkyl radical is linear and has from 1 to 5 carbon atoms. The aromatic in the arylalkyl radical can be monocyclic, bicyclic or optionally polycyclic. In the case of polycyclic aromatics. individual rings can optionally be fully or partially saturated. A preferred example of an arylalkyl radical is benzyl.

The component (A) can also comprise a plurality of structurally different subcomponents which individually correspond to the definition of the component (A).

In one possible embodiment, the composition (Z) comprises at least two components (A1) and (A2), where

component (A1) is defined according to formula (III), where R₁ is C₁-C₁₂-alkyl, preferably methyl, and R₂, R₃, R₄, R₁′, R₂′, R₃′ and R₄′ are each H; and

component (A2) is defined according to the formula (III), where R₂ is C₁-C₁₂-alkyl, preferably methyl, and R₁, R₃, R₄, R₁′, R₂′, R₃′ and R₄′ are each H.

The composition (Z) preferably comprises from 5 to 50% by weight, even more preferably from 10 to 40% by weight, particularly preferably from 15 to 30% by weight, of the component (A1), based on the total amount of the composition (Z), and

from 50 to 95% by weight, preferably from 65 to 90% by weight, even more preferably from 70 to 85% by weight, of the component (A2), based on the total amount of the composition (Z).

The provision of the composition (Z) can be effected by all methods known as suitable for this purpose to those skilled in the art.

Depending on the embodiment selected, one possibility is the targeted synthesis of the component (A), where the proportion of the component (A) in which the two NH₂ groups in β or γ positions relative to one another assume a trans configuration relative to one another is more than 50 mol %, based on the total amount of the component (A).

As an alternative, a sufficient amount of cis isomers of the component (A) in respect of the two NH₂ groups can be separated off from a composition (Z0) in which the proportion of the component (A) in which the two NH₂ groups in β or γ positions relative to one another assume a trans configuration relative to one another is not more than 50 mol % based on the total amount of the component (A) so as to give the composition (Z).

The removal of the cis isomers can be carried out by all methods known as suitable for this purpose to those skilled in the art.

For example, the removal can, in some embodiments, be carried out by chromatographic methods such as column chromatography or preparative high performance liquid chromatography (HPLC). The technical parameters required in each case when using the chromatographic methods mentioned are dependent on the specific compounds used. The procedure for determining these in a routine manner corresponds to the general technical knowledge of a person skilled in this field.

As an alternative, removal of the cis isomers by distillation is also possible.

In one embodiment of the process of the invention for preparing a cyclic isocyanate (B) in which the composition (Z) comprises the component ((A1) according to the above definition, where R₁ is methyl, and the component (A2), where R₂ is methyl, the provision of the composition (Z) is effected by distillation of a composition (Z0) in the presence of an auxiliary which has at least one alcohol group. The composition (Z0) comprises, in respect of the NH₂ groups, both cis and trans isomers of the components (A1) and (A2). The proportion of the trans isomers of (A1) and (A2), based on their NH₂ groups, is, however, not more than 50 mol % of the total amount of (A1) and (A2). The provision of the composition (Z) for this embodiment is disclosed in detail in EP 14194717, filed on Nov. 25, 2014.

In step b), the composition (Z) is reacted with phosgene to give a composition (ZP). The composition (ZP) comprises at least one cyclic isocyanate (B).

The reaction of the composition (Z) with phosgene in the gas phase or the liquid phase, preferably in the liquid phase, can be carried out by all methods known to those skilled in the art.

The reaction can be carried out in the presence of a solvent (L).

The solvent (L) is preferably inert toward phosgene under the reaction conditions of step b).

The solvent (L) is even more preferably selected from the group consisting of dichlorobenzene (DCB), chlorobenzene, THF, toluene, methylene chloride, chlorotoluene and xylene.

The solvent is particularly preferably selected from the group consisting of dichlorobenzene (DCB), chlorobenzene and chlorotoluene.

The composition (Z) is preferably added a solution in a solvent (L) to the reaction solution.

In one embodiment, the phosgene is placed in a reaction vessel in step b) and the composition (Z) is added as a solution in the solvent (L).

The composition (Z) is preferably reacted with the phosgene in step b) at a temperature of from 25 to 400° C., even more preferably from 30 to 300° C., particularly preferably from 40 to 200° C. and very particularly preferably from 40 to 150° C.

The reaction in step b) is preferably carried out at a pressure of from 0.5 to 50 bar.

In step b), the composition (Z), optionally dissolved in a solvent (L), is preferably added at a dropwise addition rate of from 0.2 to 100 ml/min, even more preferably from 0.4 to 40 ml/min, particularly preferably from 0.6 to 20 ml/min and very particularly preferably from 0.65 to 4 ml/min, to the reaction solution.

The proportion of the composition (Z) which is dissolved in the solvent (L), based on the concentration of the composition (Z) in the reaction mixture after complete addition of the composition (Z) to the reaction mixture, is preferably from 0.1 to 20% by weight, even more preferably from 0.5 to 6% by weight and particularly preferably from 0.8 to 2% by weight. Here, the term “reaction mixture” refers to the reaction solution in the reaction vessel in which the starting materials react.

The process of the invention can comprise a further step c).

In the optional step c), an isocyanate mixture (M) comprising at least one cyclic isocyanate (B) having at least two isocyanate groups is obtained by purification of the composition (ZP).

For the purposes of the invention, purification means that the at least one cyclic isocyanate (B) is at least partly separated from by-products, the starting materials and the solvent (L) until an isocyanate mixture (M) whose minimum content of the at least one cyclic isocyanate (B) conforms to the minimum content defined below is obtained. The proportion of the cyclic isocyanate (B) in the total amount of the isocyanate mixture (M) is preferably at least 80% by weight, more preferably at least 90% by weight, very particularly preferably at least 95% by weight and in particular at least 98% by weight.

Accordingly, the composition (ZP) preferably comprises less than 80% by weight, even more preferably less than 70% by weight, very particularly preferably less than 50% by weight, of the at least one cyclic isocyanate (B), based on the total amount of the composition (ZP).

The purification of the composition (ZP) can be carried out by all methods known to those skilled in the art.

The isocyanate mixture (M) is preferably obtained by distillation of the composition (ZP).

The distillation can be carried out by all methods which are known to those skilled in the art and are, in respect of the respective embodiment, industrially tried and tested. The pressure to be selected and the temperature to be used are dependent on the specific compounds to be distilled. The methods of determining these parameters correspond to the general technical knowledge of a person skilled in the art.

The distillation can, for example, be carried out, inter alia, on a rotary evaporator, in a distillation column, by bulb tube distillation or short path distillation.

The distillation can also be carried out in a plurality of steps by means of one distillation technique or a combination of various distillation techniques until the composition (ZP) has the above minimum content of the cyclic isocyanate (B) and can therefore be designated as isocyanate mixture (M).

The process of the invention can comprise a further step d). In this step d), the polymerization of the at least one cyclic isocyanate (B) is carried out using the isocyanate mixture (M) from step c) together with at least one further component (K) which has at least one amino and/or hydroxyl group and/or water to form polyurethanes or polyureas.

The polymerization can be carried out by ail methods which are known as suitable for this purpose to those skilled in the art. The polymerization can, for example, be carried out according to the procedure described in EP 0 792 899 A, EP 0792 900 A1 and EP 0 729 991 A1.

The at least one component (K) preferably has at least two hydroxyl groups, at least two amino groups or at least one amino group and at least one hydroxyl group.

Possible components (K) are, for example, compounds selected from the group consisting of ethylenediamine, 1,2- and 1,3-diaminopropane, 1,6-diaminohexane, 1,3-diamino-2,2-dimethylpropane, isophoronediamine, 1,3- and 1,4-diaminohexane, 4,4′-diaminodicyclohexylmethane, 2,4- and/or 2,6-diamino-1-methylcyclohexane, 4,4′-diamino-3,3′-dimethyldicyclohexylmethane , 1,4-bis(2-aminoprop-2-yl)cyclohexane, 3,3′-dimethyl-4,4′-diaminodicyclohexylmethane, hydrazine, hydrazides and any mixtures of such diamines and hydrazines; higher-functionality polyamines such as diethylenetriamine, triethylenetetramine, dipropylenetriamine, tripropylenetetramine, hydrogenated addition products of acrylonitrile onto aliphatic or cycloaliphatic diamines, preferably corresponding adducts of an acrylonitrile group and one molecule of a diamine, e.g. hexamethylenepropylenetriamine. tetramethylenepropylenetriamine, isophoronepropylenetriamine, 1,4- or 1,3-cyclohexanepropylenetriamine or any mixtures of such polyamines.

Furthermore, it is possible to use, for example, compounds selected from the group consisting of ethylene glycol, propylene glycol, 1,4-butanediol, 1,6-hexanediol, glycerol, trimethylolpropane, the isomeric hexanetriols or pentaerythritol and addition products of propylene oxide and/or ethylene oxide onto these compounds as component (K).

The polymerization can be carried out in the presence of a solvent (L2) which is inert, in particular toward isocyanate groups, under the conditions of the polymerization.

In the context of the polymerization, water is not counted as solvent (L2).

Preference is given to using an aprotic solvent as solvent (L2).

Particular preference is given to using solvents selected from the group consisting of dichlorobenzene (DCB), chlorobenzene, THF, methylene chloride, chlorotoluene, N-methylpyrrolidone, diethylene glycol dimethyl ether, methyl ethyl ketone, methyl isobutyl ketone, acetone, toluene, xylene, butyl acetate and methoxypropyl acetate as solvent (L2).

Very particular preference is given to using solvents selected from the group consisting of N-methylpyrrolidone, diethylene glycol dimethyl ether, methyl ethyl ketone, methyl isobutyl ketone, acetone, toluene, xylene, butyl acetate and methoxypropyl acetate as solvent (L2).

The polymerization in step d) can be carried out in the presence of at least one catalyst. These catalysts are preferably tertiary amines and/or tin compounds. This catalyst is even more preferably triethylamine and/or a tin compound selected from the group consisting of tin(II) octoate, dibutyltin oxide and dibutyltin dilaurate.

The following examples illustrate the invention.

### EXAMPLE 1

Composition (Z): 64% by weight of 1-methyl-2,4-diaminocyclohexane and 36% by weight of 1-methyl-2,6-diaminocydohexane, 74% by weight of trans isomers and 26% by weight of cis isomers with regard to the position of the NH₂ groups relative to one another, based on the total amount of 1-methyl-2,4-diaminocyclohexane and 1-methyl-2,6-diaminocyclohexane.

395 g of dichlorobenzene are heated to a temperature of 43° C. (measured in the reaction solution). A total of 35 g of phosgene is passed into this. After introduction of only 16 g of phosgene, boiling under reflux is observed and the dropwise addition of a solution of 5 g of the composition (Z) in 100 g of dichlorobenzene over a total of 1 h and 10 minutes is commenced, with the temperature in the reaction solution being from 52 to 58° C. In parallel to the addition of the composition (Z), the remaining 19 g of phosgene are passed in. After completion of the dropwise addition, the mixture is stirred at 55° C. for a further 1 hour. After completion of the dropwise addition, the concentration of the composition (Z) in the reaction solution is 1% by weight. The temperature in the reaction solution is subsequently set to from 126 to 131° C. and the solution is stirred at this temperature for 1 hour and 25 minutes. The reaction mixture is subsequently stirred at 20° C. for 15 hours and 30 minutes. The reaction solution is then again stirred at from 131 to 134° C. for 2 hours 30 minutes. Next, nitrogen is passed through the reaction solution for 21 hours at a temperature of 80° C. in the reaction solution. After this time, the solution is again heated at 160° C. for 3 hours and the mixture is completely evaporated at 85° C. and up to 7 mbar on a rotary evaporator. This gives 6.9 g of the composition (ZP).

6.4 g of the composition (ZP) are further purified by bulb tube distillation to give the isocyanate mixture (M). In the specific case of the examples and comparative examples 1 to 3 presented here, the isocyanate mixture (M) distils at 200° C. at a pressure of 0.86 mbar. 5.0 g of distillate and 0.99 g of distillation residue are obtained, corresponding to a percentage loss based on the amount of distilled composition (ZP) of 14%.

The comparative examples 1 to 3 and the further examples 2 and 3 as per table 1 were carried out in a manner analogous to example 1, but with the deviations in the ratio of the trans and cis isomers in the composition (Z) and/or varying concentrations of the composition (Z) in the reaction solution as indicated in table 1. The proportions of 1-methyl-2,4-diaminocyclohexane and 1-methyl-2,6-diaminocyclohexane in examples 2 and 3 correspond to those in example 1. In the case of comparative examples 1 to 3, the proportion of 1-methyl-2,4-diaminocyclohexane is 85% by weight and that of 1-methyl-2,6-diaminocyclohexane is 15% by weight.

TABLE 1

| | | Concentration of | |
|---|---|---|---|
| | trans/cis isomer | the composition (Z) | |
| | ratio with regard to | in the reaction | Weighed-out distillation |
| | the NH₂ groups of | solution in % by | residue after bulb tube |
| | the component (A) | weight after | distillation of the |
| | in the | completion of the | composition |
| | composition (Z) | dropwise addition | (ZP)/percentage loss |
| Comparative | 35% trans/65% | 1% | 1.3 g/26% |
| example 1 | cis | | |
| Comparative | 35% trans/65% | 5% | 4.64 g/51.02% |
| example 2 | cis | | |
| Comparative | 35% trans/65% | 10% | Mixture becomes |
| example 3 | cis | | completely solid |
| Example 1 | 74% trans/26% | 1% | 0.99 g/15% |
| | cis | | |
| Example 2 | 74% trans/26% | 5% | 4.2 g/23% |
| | cis | | |
| Example 3 | 74% trans/26% | 10% | 2 g/39% |
| | cis | | |

A higher proportion of distillation residue and thus a higher loss of product during the purification by bulb tube distillation correlates with increasing formation of high-boiling by-products during the reaction of the composition (Z) with phosgene in step b), which remain in the distillation residue.

The results show that the formation of high-boiling by-products is significantly reduced (examples 1 to 3) when using a composition (Z) in which the proportion of the trans isomers of the component (A), with regard to the position of the NH₂ groups relative to one another, makes up more than 50 mol % of the total amount of the composition (Z) compared to examples in which this condition is not satisfied (comparative examples 1 to 3).

In addition, the results indicate that the formation of the high-boiling by-products additionally increases with increasing concentration of the composition (Z) in the reaction solution.

1. A process for preparing a cyclic isocyanate (B), the process comprising reacting a composition (Z) with phosgene to give a composition (ZP) comprising at least one cyclic isocyanate having two isocyanate groups,
wherein:
the composition (Z) comprises at least one component (A) comprising at least one cycloalkane ring having at least 4 ring carbons;
the cycloalkane ring has two NH₂ groups in β or γ positions relative to one another as substituents; and
in more than 50 mol % of the total amount of component (A), the two NH₂ groups in β or γ positions relative to one another assume a trans configuration relative to one another.

2. The process according to claim 1, wherein the at least one component (A) is a compound having the formulae (I), (II), (III), (IV), (V) or (VI): 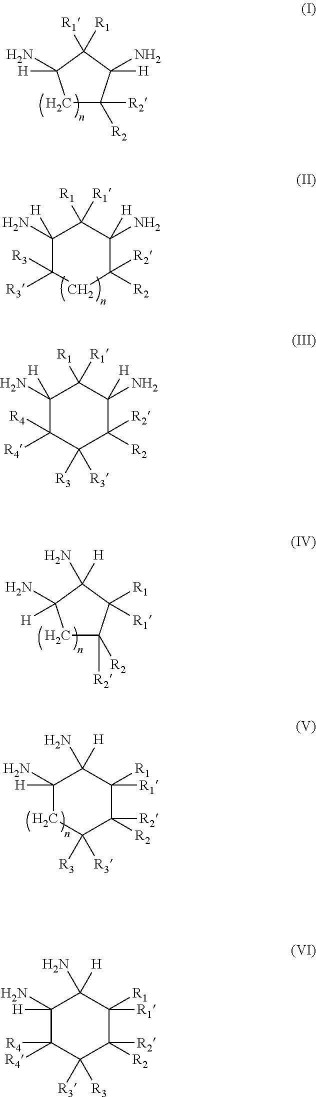
wherein:
n is from 0 to 10;
R₁, R₁′, R₂, R₂′, R₃, R₃′, R₄, R₄′, are each H, C₁-C₁₂-alkyl, C₂-C₁₀-alkenyl, aryl, arylalkyl or —OR₅; and
R₅ is C₁-C₁₂-alkyl,
where the substituents R₁, R₁′, R₂, R₂′, R₃, R₃′, R₄, R₄′ are identical or are selected independently of one another.

3. The process according to claim 2, wherein:
the composition (Z) comprises at least two components (A1) and (A2);
component (A1) is defined according to formula (III), where R₁ is C₁-C₁₂-alkyl, and R₂, R₃, R₄, R₁′, R₂′, R₃′, and R₄′ are each H; and
component (A2) is defined according to formula (III) where R₂ is C₁-C₁₂-alkyl, preferably methyl, and R₁, R₃, R₄, R₁′, R₂′, R₃′ and R₄′ are each H.

4. The process according to claim 3, wherein the composition (Z) comprises:
from 5 to 50% by weight of the component (A1), based on the total amount of the composition (Z); and
from 50 to 95% by weight of the component (A2), based on the total amount of the composition (Z).

5. The process according to claim 1, wherein:
i) in more than 60 mol %, based on the total amount of component (A), of the at least one component (A) the two NH₂, groups in β or γ positions relative to one another assume a trans configuration relative to one another, or
ii) in from 60 to 99 mol %, based on the total amount of component (A), of the at least one component (A) the two NH₂ groups in β or γ positions relative to one another assume a trans configuration relative to one another.

6. The process according to claim 1, wherein the reacting occurs in the liquid phase.

7. The process according to claim 6, wherein at least one of the following conditions is satisfied:
i) in the reacting, the phosgene is placed in a reaction vessel and the composition (Z) is added as a solution in a solvent (L);
ii) in the reacting, the composition (Z) is reacted with the phosgene at a temperature of from 25 to 400° C.; and
iii) a proportion of the composition (Z) which is dissolved in the solvent (L), based on the concentration of the composition (Z) in the reaction mixture after complete addition of the composition (Z) to the reaction mixture, is from 0.1 to 20% by weight.

8. The process according to claim 6, wherein the liquid phase comprises at least one solvent selected from the group consisting of dichlorobenzene (DCB), chlorobenzene, THF, toluene, methylene chloride, chlorotoluene and xylene.

9. The process according to claim 1, further comprising isolating an isocyanate mixture (M) comprising at least one cyclic isocyanate (B) which has at least two isocyanate groups by purification of the composition (ZP).

10. The process according to claim 9, wherein at least one of the following conditions is satisfied:
i) the proportion of the component (A) makes up at least 90% by weight of the total amount of the composition (Z); and
ii) the proportion of the cyclic isocyanate (B) makes up at least 80% by weight of the total amount of the isocyanate mixture (M).

11. The process according to claim 3, wherein: characterized in that
R₁ in component (A1) is methyl;
R₂ in component (A2) is methyl;
the composition (Z) is obtained by distilling a composition (Z0) in the presence of an auxiliary which has at least one alcohol group; and,
the composition (Z0) comprises, with regard to the NH₂ groups, both cis and tans isomers of the components (A1) and (A2), but a proportion of the trans isomers of (A1) and (A2), based on the NH₂ groups thereof, is not more than 50 mol %, based on the total amount of (A1) and (A2).

12. The process according to claim 9, further comprising polymerizing the at least one cyclic isocyanate (B) with the isocyanate mixture (M) from to isolating together with at least one further component (K) which has at least one amino group, hydroxyl group, water, or a mixture thereof, to form at least one polyurethane or at least one polyurea.

13. The process according to claim 1, wherein the cycloalkane ring of the at least one component (A) has two NH₂ groups in γ positions relative to one another as substituents.

14. The process according to claim 3, wherein:
the composition (Z) comprises at least two components (A1) and (A2);
R₁ is methyl; and
R₂ is methyl.

15. The process according to claim 4, wherein the composition (Z) comprises:
from 15 to 30% by weight, of the component (A1), based on the total amount of the composition (Z); and
from 70 to 85% by weight, of the component (A2), based on the total amount of the composition (Z).

16. The process according to claim 5, wherein:
i) in more than 65 mol %, based on the total amount of component (A), of the at least one component (A) the two NH₂ groups in β or γ positions relative to one another assume a trans configuration relative to one another, or
ii) in from 65 to 95 mol %, based on the total amount of component (A), of the at least one component (A) the two NH₂ groups in β or γ positions relative to one another assume a trans configuration relative to one another.

17. The process according to claim 7, wherein the proportion of the composition (Z) which is dissolved in the solvent (L), based on the concentration of the composition (Z) in the reaction mixture after complete addition of the composition (Z) to the reaction mixture, is from 0.5 to 6% by weight.

18. The process according to claim 10, wherein:
i) the proportion of the component (A) makes up at least 95% by weight, of the total amount of the composition (Z); or
ii) the proportion of the cyclic isocyanate (B) makes up at least 90% by weight, of the total amount of the isocyanate mixture (M).
